(19) United States
(12) Plant Patent Application Publication
(10) Pub. No.: US 2005/0257303 P1
(43) Pub. Date: Nov. 17, 2005

(54) Clematis plant named 'Carnival'

(50) CLEMATIS HYBRIDA; CLEMATIS

(76) Inventor: James P. Vanlaeken, Walworth, NY (US)

Correspondence Address: Fred L. Denson, Attorney, 14 East Main Street, PO Box 801, Webster, NY, 14580, US

(21) Appl. No.: 10/847,050

(22) Filed: May 17, 2004

(51) Int. Cl. E05B 1/00; E05B 7/00; B65D 25/28; A47J 45/00; A47B 95/02; A01H 5/00

(52) U.S. Cl. PLT/228

(57) ABSTRACT

A new variety of clematis plant named ‘Carnival’. The variety is characterized by its unique color combination, having a medium lavender blue tepal with a dark lavender bar. The bright yellow stamens further distinguish the flowers.

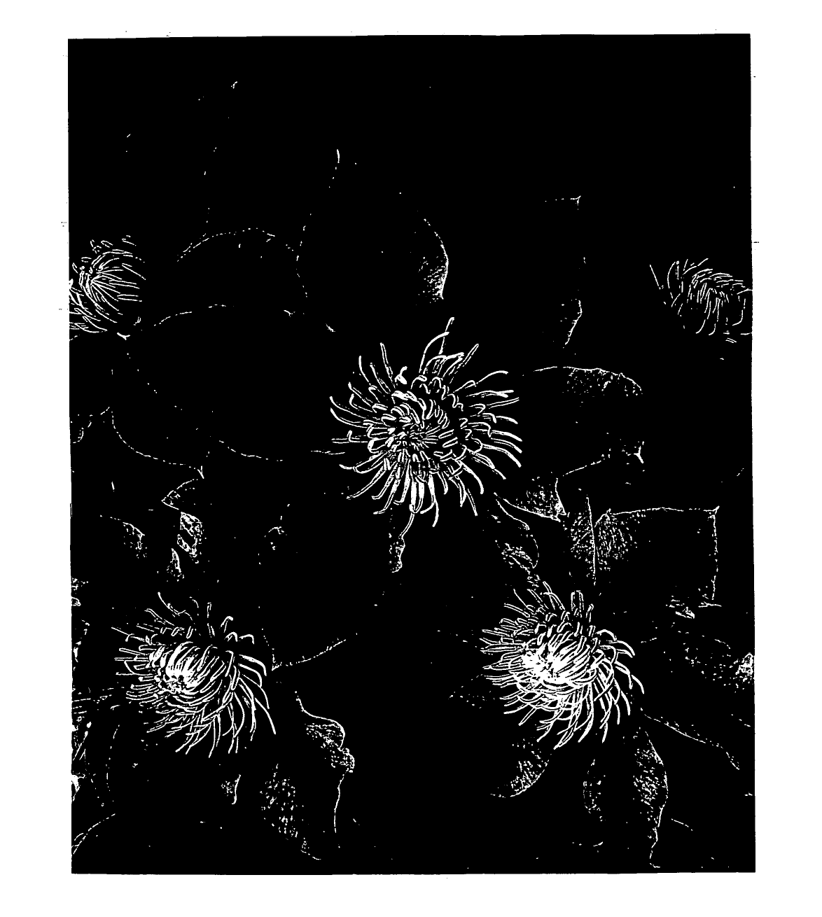

### BOTANICAL CLASSIFICATION

Clematis Species

### CULTIVAR DESIGNATION

‘Carnival’

### BACKGROUND OF THE NEW VARIETY

The present invention relates to a new and distinct variety of hybrid clematis plant known by the cultivar name ‘Carnival’.

The new variety was discovered in a controlled planting of cultivated plants in a clematis nursery in Walworth, N.Y. in 1999. Plants were asexually propagated and observed over the next two years. In 2002, an offspring was selected and planted in an outside garden area and observed. Propagation was done in Walworth, N.Y. Asexual propagation by vegetative cuttings has shown that the propagules are identical to the original plant in all distinguishing characteristics. The stability of the variety is evidenced by the distinguishing characteristics remaining true to form from generation to generation. The parentage of the variety is a cross of C. General Sikorski and C. Barbara Jackman.

### SUMMARY OF THE INVENTION

Among the features which distinguish the new variety from other presently available and known commercial clematis cultivars are the unique color of the flower. The flower has medium lavender blue tepals with a dark lavender bar that typically extends along its center longitudinal axis. The large bright yellow stamens further distinguish the flowers.

### RELEVANT PRIOR ART

The new variety is somewhat similar in appearance to Clematis ‘Dr. Ruppel’ variety as shown in the Spring Valley Greenhouse, Inc. catalogue (4^{th} Edition) at page 13. While they both have slightly wavy edges, the Carnival variety has a smaller flower and a striking blue color with a deeper purplish bar. They both have large showy stamens, however, Carnival stamens are bright yellow whereas the Dr. Ruppel variety has a brown tinge to the tips.

### BRIEF DESCRIPTION OF THE PHOTOGRAPH

The accompanying photograph illustrates the overall appearance of the new variety. The flowering and foliage colors as shown are, within the limitations of color photography, accurate and true reproductions of the actual colors of the new variety. The photographic view is a front elevation of typical Clematis Carnival.

### BOTANICAL DESCRIPTION

The following information is based on a three year old plant grown in a 5 gallon container in a covered greenhouse in Walworth, N.Y. Color references are based on using the PANTONE® matching system.
I. Plant:
   A. Form.—upright climbing or spreading
   B. Growth.—vigorous, vining 1. Height: approximately 6 feet to 8 feet 2. Diameter/Spread: approximately 2 feet to 3 feet.
   C. Foliage.—deciduous, compound-trifoliate, opposite 1. Size, typical and observed: a. compound leaf length 2.5 inches, width 5 inches b. individual leaflets length 2.25 inches, width ⅞ inch 2. Quantity per stem: typical and observed about 45 3. Color a. upper surface: dark green (PANTONE® #371C) b. lower surface: medium green (PANTONE® #377U) 4. Shape: normally three leaflets in pinnate form; each leaflet is cordate shaped 5. Texture a. upperside: glabrous veined, b. underside: ribbed 6. Petiole a. color: (PANTONE® 497C) b. length, typical and observed: 1 inch c. diameter, typical and observed: 3/64 inch.
   D. Wood.—1. New shoots a. color: light green (PANTONE® #575C) b. bark: smooth c. internodes: cylindrical, approximately 3 inches in length and 1/16 inch in diameter 2. Old Wood a. color: light brown (PANTONE® #175C) b. bark: smooth
   E. Hardiness.—US Zones 4 through 8.
   F. Disease.—1. subject to any disease that normally affects clematis a. wilt b. botrytis 2. has shown resistance to powdery mildew.
   G. Pests.—subject to any pests that normally affect clematis.
II. Flower:
   A. Blooming habit.—recurrent, May, June and September based on climate in Rochester, N.Y. area.
   B. Bud.—1. Size: typical and observed from about ⅜ inch to about ¾ inch in length and about 3/16 inch to ⅜ inch diameter 2. Form: elliptic to ovate 3. Color: green/grayish white (PANTONE® #465C).
   C. Stem.—1. Length: approximately 1 inch 2. Aspect: vertical 3. Strength: erect 4. Color: green (PANTONE® #378C)
   D. Bloom.—1. Size: typical and observed from about 3½ inches to about 6 inches in diameter with a depth of ½ inch 2. Form: round, very symmetrical with 4, 5 or 6 tepals of equal size slightly overlapping. 3. Color upper surface: edges of tepals are lavender blue (PAN-TONE® #259U) with a darker bar (PANTONE® 248C) 4. Color lower surface: Outer edge same as upper surface (PAN-TONE® #259U); center bar is whitish with a hint of grey (PANTONE® #427C) 5. Characteristics: borne on 3 inch to 4½ inch stems 6. Quantity: 1 bloom per stem 7. Lastingness: about 3 to about 6 days as a cut flower and about 7 to about 10 days on the plant.
   E. Tepals.—1. Form: obovate in shape starting 3/16 inch wide at base, expanding halfway up to 1⅛ inches at its widest point and then tapering down to a pointed apex, the typical and observed overall tepal length being about 2 inches. 2. Arrangement: regularly arranged, slightly overlapping at the base 3. Fragrance: none 4. Color: (see II D 3 above).
III. Reproductive organs:
   A. Stamens, filaments and anthers.—1. Arrangement: regularly arranged 2. Stamens: typical and observed, 65 to 70 per bloom, (PANTONE® #461C), length 9/16 inch 3. Anther: (PANTONE® #463C), length 3/16 inch 4. Pistil: typical and observed, from about 30 to 35 per bloom, ⅜ inch in length.
   B. Pollen.—1. Color: off white (PANTONE® C) 2. Amount: Scarce.
   C. Styles.—bunched, even, average length.
   D. Stigmas.—1. Color: greenish-white (PANTONE® #365C).
   E. Seed, typical and observed.—1. Diameter: ⅛ inch 2. Thickness: 1/32 inch 3. Color: (PANTONE® #463U) 4. Shape: round 5. Quantity per bloom: 8 to 15 6. Description: ¾ inch long persistent style which is clothed with 1/32 inch long silky hairs.

1. A new and distinct variety of clematis plant named ‘Carnival’ as shown and described herein.
